(19) United States
(12) Patent Application Publication
(10) Pub. No.: US 2025/0021705 A1
(43) Pub. Date: Jan. 16, 2025

(54) INFORMATION PROCESSING DEVICE, INFORMATION PROCESSING METHOD, AND NON-TRANSITORY COMPUTER READABLE MEDIUM

(71) Applicants: ENEOS Corporation, Tokyo (JP); Preferred Networks, Inc., Tokyo (JP)

(72) Inventors: Kota MATSUMOTO, Tokyo (JP); Taku WATANABE, Tokyo (JP); Takafumi ISHII, Tokyo (JP); Ryohto SAWADA, Tokyo (JP)

(73) Assignees: ENEOS Corporation, Tokyo (JP); Preferred Networks, Inc., Tokyo (JP)

(21) Appl. No.: 18/900,321

(22) Filed: Sep. 27, 2024

Related U.S. Application Data
(63) Continuation of application No. PCT/JP2023/010056, filed on Mar. 15, 2023.

(30) Foreign Application Priority Data
Mar. 31, 2022 (JP) 2022-060943

(51) Int. Cl. G06F 30/10; G16C 20/10

(52) U.S. Cl. CPC G06F 30/10; G16C 20/10

(57) ABSTRACT

An information processing device includes one or more memories; and one or more processors. The one or more processors are configured to search for a reaction path by using one or more trained models that, when receiving an input of a three-dimensional arrangement of two or more atoms forming a molecule, output a physical quantity regarding the molecule.

### CROSS REFERENCE TO RELATED APPLICATIONS

This application is continuation application of International Application No. JP2023/010056, filed on Mar. 15, 2023, which claims priority to Japanese Application No. 2022-060943, filed on Mar. 31, 2022, the entire contents of which are incorporated herein by reference.

### FIELD

This disclosure relates to an information processing device, an information processing method, and a non-transitory computer readable medium.

### BACKGROUND

A reaction path search and a transition state search are important to obtain information on transition states, activation parameters, rate constants, or the like in intermolecular reaction paths. In the reaction path search, it is relatively easy to search for a stable structure, but it is usually difficult to find a saddle point that can be a reaction path from this stable structure. The reaction path search uses, for example, a stationary point search or a Nudged Elastic Band (NEB) method, but the former does not succeed in the search unless executing the search from the neighborhood of the saddle point, and in the latter, it is prerequisite to obtain rough reaction paths beforehand. Techniques such as an Anharmonic Downward Distortion Following (ADDF) method and an Artificial Force Induced Reaction (AFIR) method, or Global Reaction Route Mapping (GRRM: registered trademark) that uses these methods in combination can solve these problems.

Though the use of the aforesaid methods enables an automated reaction path search, the ADDF method and the AFIR method entail high calculation costs, and the size of molecules regarding which they can execute the calculation and the number of molecules regarding which they can execute the search are limited. This is because these methods need to calculate energy, force, or Hessian by a first-principles calculation an enormous number of times while moving atomic nuclei. These methods require executing the computation also on structures distant from a stable molecule and thus are difficult to apply to a classical force field. In the ADDF method, a direction in which an anharmonic downward distortion is large is set as a molecular deformation direction, but the calculation of the anharmonic downward distortion by the first-principles calculation costs very high. Accordingly, as the price of the use of the high-speed method, a worry about a search failure accompanies. Further, in the AFIR method, it is difficult to decide artificial force parameters, and it is also difficult to determine whether the accurate reproduction of a reaction has succeeded after the end of the computation.

### BRIEF DESCRIPTION OF THE DRAWINGS

FIG. 1 to FIG. 5 are flowcharts illustrating processing according to embodiments.

FIG. 6 is a block diagram illustrating an example of hardware implementation according to the embodiments.

### DETAILED DESCRIPTION

According to one embodiment, an information processing device includes one or more memories; and one or more processors. The one or more processors are configured to search for a reaction path by using one or more trained models that, when receiving an input of a three-dimensional arrangement of two or more atoms forming a molecule, output a physical quantity regarding the molecule.

Embodiments of this disclosure will be hereinafter described with reference to the drawings. The drawings and the description of the embodiments are presented by way of example only and are not intended to limit the present invention.

First, an ADDF method and an AFIR method used for an automated search in this disclosure will be briefly described, and then, embodiments for implementing these methods will be described in detail with reference to the drawings.

### (ADDF Method)

The ADDF method is a method to find a transition state by deforming a molecule in a direction in which potential energy becomes small as compared with harmonic approximation, that is, in a direction in which an anharmonic downward distortion (ADD) is minimum. The descriptions are presented by way of example only and are not restrictive in the embodiments in this disclosure.

In both a first-principles calculation and a trained model, energy E is calculated as a function of an atomic nucleus coordinate X.

$$E = f ( {x}_{1} , {x}_{2} , {x}_{3} , … , {x}_{3 N} ) \qquad ( 1 )$$

Here, N is the number of atoms.

Therefore, whichever of the first-principles calculation and the trained model is used, it is possible to search for a structure where energy becomes minimum, by any of various optimization methods such as, for example, a steepest descent, a conjugate gradient method, and a Broyden-Fletcher-Goldfarb-Shanno (BFGS) algorithm. In other words, it is possible to search for x0=(x0₁, x0₂, x0₃, . . . , x0_{3N})^{T} which is the atomic nucleus coordinate of a stable structure.

At x0, the first derivative of the energy with respect to the coordinate, that is, force, is 0, and therefore, harmonic approximation energy at x=(x₁, x₂, x₃, . . . , x_{3N})^{T} in the neighborhood of x0 is as follows.

$$E ≃ {\sum }_{i = 1}^{3 N} {\sum }_{j = 1}^{3 N} {H}_{i , j} Δ {x}_{i} Δ {x}_{j} = Δ {x}^{T} H Δ x = {E}_{harmonic} \qquad ( 2 )$$

Here, Δx=x−x0, and H is a Hessian matrix (Hessian) at x0. Further, T represents the transposition of a vector or a matrix.

$${H}_{i , j} = \frac{{\partial }^{2} E}{\partial {x}_{i} \partial {x}_{j}} \qquad ( 3 )$$

Consider coordinate-transforming Δx to y using an orthogonal matrix R.

$$y = R Δ x , \qquad ( 4 )$$

$${y}^{T} = Δ {x}^{T} {R}^{T}$$

In this case, the harmonic approximation energy can be represented as follows.

$$Δ {x}^{T} H Δ x = {y}^{T} {RHR}^{T} y \qquad ( 5 )$$

Since the Hessian matrix H is a symmetric matrix, if R is appropriately selected, RHRT becomes a diagonal matrix D.

$$Δ {x}^{T} H Δ x = {\sum }_{i = 1}^{3 N} {D}_{ii} {y}_{i}^{2} \qquad ( 6 )$$

Further, coordinate transforming of zᵢ=Dᵢᵢ^{1/2}yᵢ gives the following equation.

$$Δ {x}^{T} H Δ x = {\sum }_{i = 1}^{3 N} {z}_{i}^{2} \qquad ( 7 )$$

On thus obtained hypersphere within a 3N dimension, a direction in which actual energy not being harmonic approximation becomes minimum is a direction where a transition state is present, and by deforming the molecule in this direction, the transition state can be reached.

Various methods are usable for searching for the energy minimum direction under a constraint condition Σ_{zi}²=r². For example, the Lagrange's method of undetermined multipliers is also usable. Further, in polar coordinate representation where r is fixed as follows, θ₁ to θ_{3N-1} may be optimized.

$${x}_{1} = r cos {θ}_{1} \qquad ( 8 )$$

$${x}_{2} = r sin {θ}_{1} cos {θ}_{2}$$

$${x}_{3} = r sin {θ}_{1} sin {θ}_{2} cos {θ}_{3}$$

$${x}_{4} = r sin {θ}_{1} sin {θ}_{2} sin {θ}_{3} cos {θ}_{4}$$

$$⋮$$

$${x}_{3 N - 1} = r sin {θ}_{1} … cos {θ}_{3 N - 1}$$

$${x}_{3 N} = r sin {θ}_{1} … sin {θ}_{3 N - 1}$$

The method of searching for the direction in which the molecule is to be deformed, starting from the energy minimum structure has been described, but once the molecule is deformed, the energy is not in the minimum state anymore. In this case, the harmonic approximation energy includes a first-order term with respect to Δx.

$$E ≃ {\sum }_{i = 1}^{3 N} {\sum }_{j = 1}^{3 N} {H}_{i , j} Δ {x}_{i} Δ {x}_{j} + {\sum }_{j = 1}^{3 N} {G}_{i} Δ {x}_{i} = Δ {x}^{T} H Δ x + G \cdot Δ x = {E}_{harmonic} \qquad ( 9 )$$

In this case, however, it is also possible to decide the molecular deformation direction by similarly handling Δx^{T}HΔx.

Continuing the molecular deformation in the direction in which the energy difference from the harmonic approximation becomes minimum makes it possible to find the transition state which is a saddle point of the energy. Note that, if the neighborhood of the saddle point instead of the saddle point itself of the energy can be reached, it is possible to more easily reach the saddle point by executing a search such as a search for a stationary point by a Newton method.

If the transition state which is the saddle point of the energy can be found, it is possible to obtain a reaction path from this by energy minimization. A method of automatically searching for the reaction path by calculating energy or a Hessian matrix using a trained model will be described as a first embodiment.

In the case where the trained model outputs, from the molecular deformation direction Δx, a difference from the harmonic approximation on a hypersurface where the harmonic approximation energies become equal, it is possible to search for the transition state by deforming the molecule in the direction where this energy difference value becomes minimum. A method to search for this transition state using the trained model will be described as a second embodiment.

In the ADDF method, it takes time to calculate the Hessian matrix (the second derivative of the energy) and decide the molecular deformation direction based on the result. Here, it is possible to reduce the number of times the Hessian matrix is computed by once finding the molecular deformation direction and thereafter continuing the molecular deformation in this direction along with the calculation of force (the first derivative of the energy). The Hessian matrix computation costs high and takes time. Therefore, reducing the number of times this computation is executed achieves a higher speed.

Further, even in the case where the molecular deformation direction is re-calculated, it is not necessarily essential to execute the high-cost Hessian matrix computation in every iteration. As one example of this method, a Hessian update method is used. The Hessian update method uses a gradient change accompanying a structural change to update a direction component along the structural change in the Hessian matrix.

Since the obtained Hessian matrix is approximate, its accuracy influences the behavior of the stationary point search calculation. At the stage where the structure is made to approach the energy stationary point, the accuracy of the Hessian matrix is not very important. On the other hand, a convergence rate from the neighborhood of the energy stationary point to the energy stationary point greatly depends on the accuracy of the Hessian matrix.

Accordingly, if the Hessian update method is used, the number of structure optimization steps increases in the immediate vicinity, but since the acquisition of the Hessian matrix by the computation is not necessary, the time required for each optimization step can be greatly reduced.

The total calculation amount is decided in view of these computations, but in many cases, the use of the Hessian update method can reduce the computation time. Another application method is to calculate an accurate Hessian matrix every several steps and use the Hessian update in the other steps.

Here, by calculating the difference using a gradient change Δgₖₗ=gₖ−gᵢ corresponding to a change Δxₖₗ=xₖ−xₗ between a structure xₖ and a structure xₗ, it is possible to obtain an updated matrix ΔHₖₗ^{DIFF}.

$$Δ {H}_{kl}^{DIFF} = \frac{Δ {x}_{kl} Δ {g}_{kl}^{T}}{Δ {x}_{kl}^{T} Δ {x}_{kl}} + \frac{Δ {g}_{kl} Δ {x}_{kl}^{T}}{Δ {x}_{kl}^{T} Δ {x}_{kl}} - \frac{Δ {g}_{kl}^{T} Δ {x}_{kl}}{Δ {x}_{kl}^{T} Δ {x}_{kl}} \frac{Δ {x}_{kl} Δ {x}_{kl}^{T}}{Δ {x}_{kl}^{T} Δ {x}_{kl}} - \frac{Δ {x}_{kl} Δ {x}_{kl}^{T}}{Δ {x}_{kl}^{T} Δ {x}_{kl}} {H}_{0}^{OLD} - {H}_{0}^{OLD} \frac{Δ {x}_{kl} Δ {x}_{kl}^{T}}{Δ {x}_{kl}^{T} Δ {x}_{kl}} + \frac{Δ {x}_{kl} Δ {x}_{kl}^{T}}{Δ {x}_{kl}^{T} Δ {x}_{kl}} {H}_{0}^{OLD} \frac{Δ {x}_{kl} Δ {x}_{kl}^{T}}{Δ {x}_{kl}^{T} Δ {x}_{kl}} \qquad ( 10 )$$

Here, H₀^{OLD} is the Hessian matrix before the update. In the case where the structure optimization calculation is executed, a matrix where Hₖₗ^{DIFF} is added to H₀^{OLD} is actually used as a Hessian matrix for the decision in the optimization step. In the right side, the first term to the third term correspond to gradient differences along Δxₖₗ, and the fourth term to the sixth term correspond to terms erasing projection parts in the Δxₖₗ direction in them.

In the structure optimization of the minimum point and the first-order saddle point, other Hessian update methods giving better results are also available, whose detailed description will be omitted.

A method using a trained model in the method of thus calculating or updating the Hessian matrix at an appropriate instant will be described as a third embodiment. As previously described, in this disclosure using the trained models, it is possible to surely execute the search without executing these computation time reductions, but executing these computation time reductions achieves a higher speed.

### (AFIR Method)

The ADDF method is a method to execute the search, starting from one molecule, while the AFIR method is a method also capable of searching for a path of a reaction of a plurality of molecules.

The Artificial Force Induced Reaction (AFIR) method induces a reaction by pressing reactant molecules against each other. For example, in the case where reactants A and B are made to react, it adds a term proportional to a distance r_{AB} between A and B to energy to thereby execute structure optimization on a potential surface where a reaction barrier is canceled.

$$F ( {r}_{AB} ) = E ( {r}_{AB} ) + α {r}_{AB} \qquad ( 11 )$$

If this is used, an optimization problem requiring the search also regarding a potential surface uphill direction, which has been difficult in the reaction path search, can be replaced with a structure optimization problem in a potential surface downhill direction. According to the AFIR method, by removing the artificial force term αr_{AB} after the end of the path calculation, it is possible to obtain an approximate reaction path and also to locate the position of the barrier.

Further, in contrast to the ADDF method, the AFIR method is capable of searching for the reaction path without executing the high-cost anharmonic downward distortion calculation.

In the application to polyatomic molecules, an artificial force function that can be represented as follows is used.

$$F ( Q ) = E ( Q ) + ρ α \frac{{\sum }_{i ∈ A} {\sum }_{j ∈ B} {ω}_{ij} {r}_{ij}}{{\sum }_{i ∈ A} {\sum }_{j ∈ B} {ω}_{ij}} \qquad ( 12 )$$

Here, E(Q) is potential energy dependent on the coordinate Q. In the case of p=1, the reactant molecules are pressed against each other by the artificial force, and in the case of p=−1, the reactant molecules are dissociated by the artificial force. rᵢⱼ is the distance between atoms i and j. rᵢⱼ is multiplied by a weighting function wᵢⱼ, and the sum is calculated in all the atoms included in the fragments A and B. Further, Ri and Rj are covalent bond radii of the atoms i and j respectively.

$${ω}_{ij} = {[ \frac{{R}_{i} + Rj}{{r}_{ij}} ]}^{p} \qquad ( 13 )$$

The calculation is not sensitive to the value of p, and it is often set to 6.0 as a given real number p. A constant α for finding the magnitude of the force is given by the following equation.

$$α = \frac{γ}{[ {2}^{- \frac{1}{6}} - {( 1 + \sqrt{1 + \frac{γ}{ε}} )}^{- \frac{1}{6}} ] {R}_{0}} \qquad ( 14 )$$

The constant γ is model collision energy and gives an approximate upper limit of a barrier that can be overcome. The model collision energy γ is set by a user. Here, as a nonlimiting example, ε=1.0061 [kj/mol] and R0=3.8164 [Å] can be set, in which case, a corresponds to average force acting on two Ar atoms from an energy minimum point up to a turning point of corresponding Lennard-Jones potential, in a normal collision of the two Ar atoms under the model collision energy γ.

A GRRM program is implemented with a Single-Component AFIR (SC-AFIR) algorithm which is an automated reaction path search method using the AFIR method. The SC-AFIR method automatically defines fragments to which the artificial force is to be applied and executes a path search by employing the AFIR method to find a new stable structure EQ. In the new EQ, the AFIR method is also used to obtain a new reaction path and EQ.

The SC-AFIR method automatically executes this iterative processing. In this automated search, not only the pressing force (p=1) but also the dissociating force (p=−1) is used, and thus it is also possible to search for a path of a reaction involving bond breaking.

Therefore, the setting of a computation condition such as the setting of appropriate γ sometimes requires user's experience. In Non-patent Document 3, 100 [kJ/mol] is given as an example of the setting of γ, which can serve as a yardstick.

A method that uses a trained model in this AFIR will be described as a fourth embodiment.

Hereinafter, the above methods will be described as embodiments which show non-limiting examples. In the embodiments, as processing, software-based information processing is concretely realized using hardware resources, for instance. An information processing device which is hardware is implemented with, for example, one or more processors (processing circuits) and one or more memories (storage circuits) connected to the processors.

The processing circuit included in the information processing device writes or reads data to/from the storage circuit at an appropriate instant. The data may be data that is necessary for the processing or is to be an input/output, or may be a program for executing the information processing.

In the following, the trained models are each a model related to Neural Network Potential (NNP), for instance. The NNP-related model is, for example, a model that, when receiving an input of information on a three-dimensional arrangement or the like of atoms, outputs energy corresponding to these atoms. The information processing device can use this model by the processing circuit referring to the storage circuit. As the NNP, MATLANTIS (registered trademark) may be used.

In the flowcharts, processes surrounded by the thick lines are, in this disclosure, processes where computation can be executed using the trained model. In the following, energy or the like is used as a physical quantity, but the physical quantity to be used can be selected as desired. Further, the processing circuit uses the models, mainly, trained models in NNP for obtaining, for example, energy, but this can also be appropriately read as trained models for obtaining a desired physical quantity.

### First Embodiment

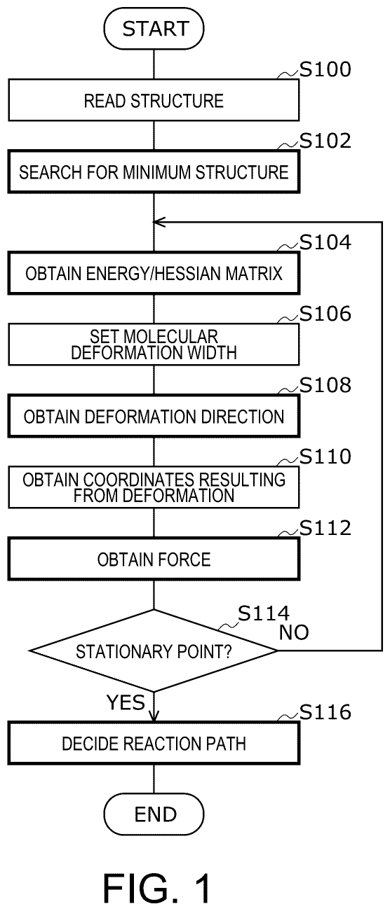

FIG. 1 is a flowchart illustrating processing of the processing circuit according to the first embodiment. In this embodiment, an example of the use of a trained model that speeds up the processing in the ADDF method will be described.

The processing circuit reads data regarding a structure of a target molecule or the like (S100). The data regarding the structure is, for example, data including the kind of atoms forming the molecule and the three-dimensional coordinates of the atoms.

The processing circuit calculates a physical quantity, for example, energy regarding the read structure, and in the case where this physical quantity is not minimum, searches for an energy minimum structure (S102). The processing circuit may use the trained model to execute this process. Note that this process is outside iteration, and therefore, not using the trained model for this process is more unlikely to constitute a bottleneck than in modeling of the other processes.

In the case where the trained model is used in S102, a model that, when receiving an input of the structure, outputs energy, that is, outputs the value of equation (1) is usable as the trained model. Further, the processing circuit can use a trained model that is trained using, as training data, a stable structure obtained by any of the above-described various optimization methods. In the case where the energy is not minimum, the processing circuit obtains, through the trained model, a stable structure x0 for the structure x read in S100.

In the case where the physical quantity obtained from the read structure x is minimum or after the structure x0 where the physical quantity is minimum is found in S102, the processing circuit sets the structure where it becomes minimum as an initial value and executes processes (S104 to S112) of searching for a transition state.

The processing circuit obtains a physical quantity from the structure (S104). The processing circuit obtains, for example, energy and a Hessian matrix as illustrated in FIG. 1. This process may be executed using a trained model. The processing circuit may obtain the energy from the structure by using the trained model and obtain the Hessian matrix by calculating the second derivative of the energy. Instead, a physical quantity from which the second derivative of the energy can be calculated may be output by the trained model, and the second derivative (Hessian matrix) of the energy may be obtained using this physical quantity. Examples of the physical quantity from which the second derivative of the energy can be calculated include a natural frequency and a reduced mass.

As another example, the processing circuit may use a trained model that, when receiving an input of the structure data, outputs the Hessian matrix. As still another example, in the case where force has been obtained, the processing circuit may obtain the Hessian matrix by taking the position derivative of this force. In and after the second iteration, by using the result of a process in later-described S112 in the previous iteration, the processing circuit is capable of shortening the time required for the Hessian matrix computation.

In ADDF, the direction in which the molecule is to be deformed, for example, a direction of translation, rotation, bending, or the like can be found. The processing circuit sets a width of this molecular deformation within a range of the maximum value of a molecular deformation displacement amount designated by a user (S106). As an initial value, one input by the user may be used. From the next iteration, a method of automatically deciding the width based on the width obtained in the previous iteration, for example, narrowing or widening the width based on the state obtained in the iteration may be employed. For the setting of the molecular deformation width, a known method may be used.

After setting the molecular deformation width, the processing circuit calculates a direction in which the difference of the energy from harmonic approximation becomes minimum, that is, a molecular deformation direction (S108). The processing circuit may calculate the difference from the harmonic approximation based on, for example, harmonic approximation energy calculated from equations (2, 3, 4, 5, 6, 7, 9), or may search for the energy minimum on a hypersphere where the harmonic approximation energies are equal, represented by equation (8). Further, the processing circuit may be configured to obtain this direction by using a trained model. The trained model used in this step may be, for example, a model that is trained using various training data obtained using equation (2) to equation (9).

The processing circuit obtains the coordinates of the atoms of the molecule deformed in the direction obtained in S108 within the width set in S106 (S110).

The processing circuit calculates force acting on the atoms by using a structure including the coordinates resulting from the deformation (S112) This step may be executed using a trained model. For example, the processing circuit may obtain the energy of the atoms by using a trained model that obtains energy from structure data, and take the position derivative (first derivative) of this energy, to thereby obtain the force. As another example, the processing circuit may obtain the force by using a trained model that obtains force from structure data.

The processing circuit determines whether or not the structure obtained in S110 is a stationary point (S114). The processing circuit may execute this determination based on, for example, whether or not the force obtained in S112 is 0. Further, a criterion for this determination by the processing circuit may be, for example, whether or not this force is equal to or less than a predetermined minute value.

In the case where the structure is not determined as the stationary point (S114: NO), the processing circuit iterates the processes from S104 by using the structure resulting from the deformation in S110.

In the case where the structure is determined as the stationary point (S114: YES), the processing circuit decides a reaction path based on the structure resulting from the deformation in S110 (S116). The processing circuit decides the reaction path by, for example, executing structure optimization in two directions from the transition state. For the processing of the structure optimization in the decision of the reaction path, the processing circuit may use a trained model. As in the process in S102, in the process in S116, which is also outside the iteration, the processing circuit may use a typical optimization method instead of using the trained model, for reasons of accuracy improvement or the like.

As described above, according to this embodiment, the information processing device is capable of executing the computation with a desired granularity by using the trained model. In the case where the trained model is not used, it is necessary to find energy by the first-principles calculation and find the first derivative (force) of this energy and the second derivative (Hessian matrix) of the energy, but due to the long time required for computing this energy, the stationary point search process where this has to be successively executed a plurality of times in the iteration requires an enormously long time.

According to this embodiment, thanks to a great increase in the energy calculation speed, it is possible to shorten the whole processing time. In addition, in the case where the trained model is capable of outputting the first derivative of the energy, it is possible to obtain the Hessian matrix by finding the first derivative of this value. This also contributes to the speed increase. Further, in the case where the trained model is capable of outputting the second derivative of the energy, the effect of increasing the speed is more prominent.

The above describes that it can be selected whether to use the trained model for each process, but this is not limiting. For example, it may be selected whether to execute the same process by iteration using the trained model or to execute it by the first-principles calculation or the like. A nonlimiting acceptable example is to execute the first-principles calculation for at least one processing step every predetermined number of iterations to attain a certain degree of accuracy. This applies not only to this embodiment but also to any of the embodiments described below.

### Second Embodiment

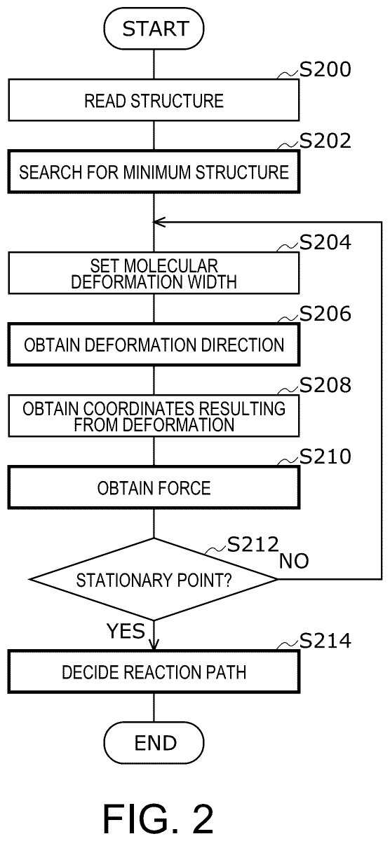

FIG. 2 is a flowchart illustrating processing of the processing circuit according to the second embodiment. In this embodiment, an example of the use of a trained model that speeds up the processing in the ADDF method will be described.

The processing circuit reads data regarding a structure of a target molecule or the like (S200). This process is the same as the process in S100 in the first embodiment.

The processing circuit calculates a physical quantity, for example, energy, regarding the read structure, and in the case where this physical quantity is not minimum, searches for an energy minimum structure (S202). This process is the same as the process in S102 in the first embodiment.

In the case where the physical quantity obtained from the read structure x is minimum or after a structure x0 where the physical quantity becomes minimum is found in S202, the processing circuit sets this structure with the minimum physical quantity as an initial value and executes processes (S204 to S210) of searching for a transition state.

The processing circuit sets a width by which the molecule is to be deformed (S204). This process is, for example, the same as the process in S106 in the first embodiment.

As another example, in this process, the processing circuit may execute the process without obtaining at least one of energy, force, or a Hessian matrix by using a trained model. The processing circuit may use a trained model that outputs anharmonicity when receiving an input of the structure, to obtain this deformation direction.

As an example, the processing circuit may use a model that outputs, from the molecular deformation direction, a difference from the harmonic approximation on a hypersurface where the harmonic approximation energies are equal. By using such a model to search for the molecular deformation direction where the energy difference becomes minimum, the processing circuit is capable of inferring the deformation direction. For the training of this trained model, it is also possible to obtain appropriate training data.

After setting the molecular deformation width, the processing circuit calculates a direction in which the energy difference from the harmonic approximation becomes minimum, that is, the molecular deformation direction (S206). This process is the same as the process in S108 in the first embodiment.

The processing circuit obtains the coordinates of the atoms of the molecule deformed in the direction obtained in S206 within the width set in S204 (S208). This process is the same as the process in S110 in the first embodiment.

The processing circuit calculates force acting on the atoms by using a structure including the coordinates resulting from the deformation (S210). This process is the same as the process in S112 in the first embodiment.

The processing circuit determines whether or not the structure obtained in S208 is a stationary point (S212). This process is the same as the process in S114 in the first embodiment.

In the case where the structure is not determined as the stationary point (S212: NO), the processing circuit iterates the processes from S204 using the structure resulting from the deformation in S208.

In the case where the structure is determined as the stationary point (S212: YES), the processing circuit decides a reaction path based on the structure resulting from the deformation in S208 (S214). Processes in and after this are the same as the processes in S114 to S116 in the first embodiment.

According to this embodiment, the information processing device is capable of executing the computation with a desired granularity by using the trained model as in the first embodiment. Moreover, according to this embodiment, it is possible to obtain data for finding the deformation direction without directly obtaining the energy or the like.

The search for the molecular deformation direction did not sometimes succeed in real time due to many computations in the interim process. According to the information processing device of this embodiment, it is possible to obtain data on anharmonicity without calculating the physical quantity such as energy, achieving a surer search. Further, since it is not necessary to obtain various physical quantities for obtaining the anharmonicity, it is possible to further speed up the method in the first embodiment.

### Third Embodiment

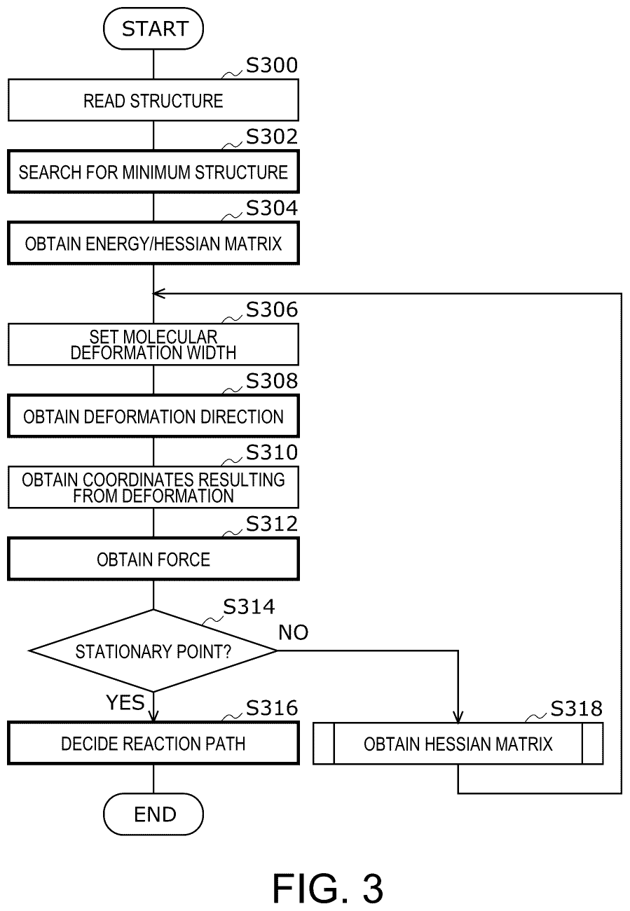

FIG. 3 is a flowchart illustrating processing of the processing circuit according to the third embodiment. In this embodiment, an example of the use of a trained model that speeds up the processing in the ADDF method will be described. The processing according to this embodiment traces the processing of ADDF that does not use a trained model, and uses the trained model in processing requiring a high computation cost.

The processes in S300 and S302 are the same as the processes in S100 and S102 in the first embodiment.

Outside the iteration, the processing circuit obtains a physical quantity, for example, energy and a Hessian matrix from the structure. (S304). The contents of the process are the same as those in S104 in the first embodiment.

In the case where the physical quantity obtained from the structure x read in S302 is minimum or after the structure x0 where the physical quantity becomes minimum is found in S302, the processing circuit executes processes (S306 to S318) of searching for a transition state, with this structure where the physical quantity becomes minimum being set as an initial value.

The processes in S306 to S316 are the same as the processes in S106 to S116 in the first embodiment respectively except a process in a branch destination from S314.

In the case where the structure resulting from the deformation is not a stationary point (S314: NO), the processing circuit shifts to a process of obtaining a Hessian matrix (S318).

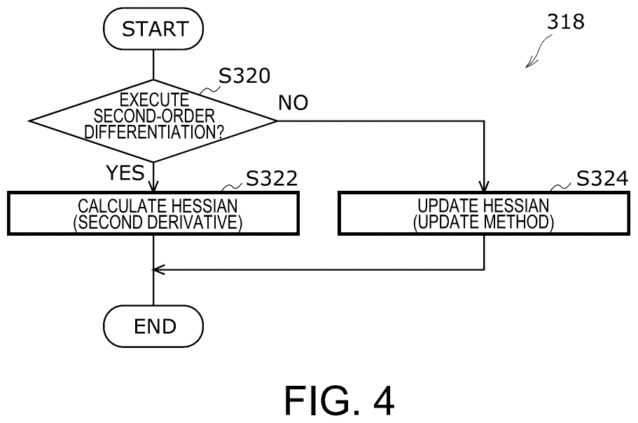

FIG. 4 is a flowchart illustrating processing of obtaining the Hessian matrix according to this embodiment.

When determining to obtain the Hessian matrix, the processing circuit determines whether to execute second-order differentiation (S320). As described above, in the case where a deformation direction is further obtained after the molecule is deformed, it is not always necessary to execute the high-cost Hessian matrix computation in every iteration.

The processing circuit determines whether to compute a high-accuracy Hessian matrix by executing the second-order differentiation or to update the Hessian matrix without executing the second-order differentiation, according to various conditions. The condition for this decision may be, for example, the number of iteration times or may be an energy difference or the like.

In the case where it is determined to execute the second-order differentiation (S320: YES), the processing circuit obtains the Hessian matrix by executing the second-order differentiation (S322). The processing circuit may use a trained model to obtain the Hessian matrix. Further, the trained model may output a physical quantity from which the second derivative of the energy can be calculated, and the second derivative (Hessian matrix) of the energy may be obtained using this physical quantity. Examples of the physical quantity from which the second derivative of the energy can be calculated include a natural frequency and a reduced mass.

For example, the processing circuit may obtain the energy from the trained model to execute the second-order differentiation, may obtain force from the trained model to execute first-order differentiation, or may obtain the Hessian matrix from the trained model.

In the case where it is not determined to execute the second-order differentiation (S320: NO), the processing circuit obtains the Hessian matrix without executing the second-order differentiation (S324). For example, the processing circuit uses a Hessian update method to update the Hessian matrix. The Hessian update method updates the Hessian matrix based on equation (10). The processing circuit may execute this process by using a trained model or execute this process without using the trained model.

As described above, according to this embodiment, the information processing device is capable of executing the computation with a desired granularity by using the trained model. In the case where the trained model is not used, it is necessary to find the energy by the first-principles calculation and find the first derivative (force) of this energy and the second derivative (Hessian matrix) of the energy, but due to the long time required for the computation on the energy, the process of the stationary point search where this has to be successively executed a plurality of times in the iteration requires an enormously long time.

According to this embodiment, by appropriately skipping the computation of the Hessian matrix, it is possible to attain a higher speed as compared with the previously described embodiments.

In the first embodiment to the third embodiment, the ADDF method has been described, but in this ADDF method, the Hessian matrix computation limits the speed. As an example, in the case where it is executed by the first-principles calculation, the time currently required by a four-core processor is about 1028 minutes and that by a 16-core processor is about 340 minutes. On the other hand, according to the embodiment of this disclosure, the required time is about 1.33 seconds without I/O included and is about 33 seconds with I/O included. Therefore, a speed about 600 times higher is achieved even under a conservative estimate.

### Fourth Embodiment

The information processing device according to this embodiment executes processing at a high speed by using a trained model in the AFIR method.

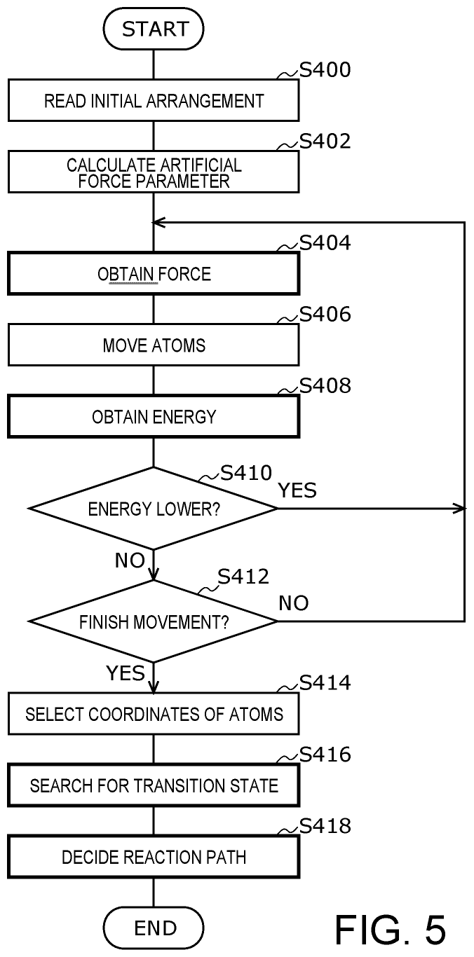

FIG. 5 is a flowchart illustrating an example of processing according to this embodiment.

The processing circuit reads an initial arrangement of molecules (S400). For example, the processing circuit reads the arrangement of the molecules of interest in the AFIR method.

The processing circuit calculates a parameter (artificial force coefficient) ωᵢⱼ in equation (12) (or a in equation (11)) of artificial force between these read molecules (S402). For example, the processing circuit obtains a weighting coefficient of the artificial force based on equation (14) or the like from model collision energy γ. The processing circuit may obtain this parameter by, for example, inferring an energy maximum point or calculating an energy difference ΔE from the starting structure.

The processing circuit may set a definition for calculating the artificial force parameter from an input value or for automatically inferring it. In the automated inference definition, from designated fragments and a force direction (attractive force or repulsive force), the processing circuit infers two reactant atoms, infers activation energy in the neighborhood of the transition state, and automatically defines artificial force necessary for the path search. Several nonlimiting examples of usable methods of deciding the artificial force parameter are as follows.

The processing circuit may decide the artificial force parameter from, for example, an input parameter.

The artificial force parameter may be inferred, using, as the input parameter, a list of at least one of a typical activation parameter, bond energy, or a reaction rate constant involved in bond formation/dissociation of the inferred two reactant atoms, which are set in advance.

The artificial force parameter may be inferred by inferring an intermediate using, as the input parameter, a pre-set typical interatomic distance in a transition state involved in bond formation/dissociation of the inferred two reactant atoms, and executing a single-point energy calculation.

The artificial force parameter may be inferred by using, as the input parameter, a pre-set typical reaction coordinate distance in the transition state involved in bond formation/dissociation of the inferred two reactant atoms, creating a structure resulting from the movement by the typical reaction coordinate distance on PES (Potential Energy Surface) where very large artificial force (about ten times or more of a value discussed in a typical chemical reaction) is applied between the designated fragments, and executing a single-point energy calculation with this point set as an inferred intermediate.

The processing circuit searches for the positions of the atoms by using the calculated artificial force parameter (S404 to S410).

The processing circuit obtains force according to equation (11) or equation (12) by using the parameter found in S402 (S404). The processing circuit may use a trained model that obtains the force from the atomic arrangement and the artificial force parameter, to execute the calculation for obtaining the force.

The processing circuit moves the positions of the atoms according to the force obtained in S404 (S406).

The processing circuit obtains energy based on a structure conforming to the positions of the atoms moved in S406 (S408). The processing circuit may use a trained model to execute the calculation for obtaining the energy.

The processing circuit determines whether or not the energy obtained in S408 is lower as compared with energy before the atoms are moved in S406 (S410). In this comparison, the processing circuit may use an energy value obtained in the previous iteration. Further, in the first iteration, the processing circuit may separately obtain energy in the state where the atoms are not moved, by using a trained model or without using the trained model.

In the case where the energy is lower (S410: YES), the processing circuit iterates the processes from S404.

In the case where the energy is not lower (S410: NO), the processing circuit determines whether to finish the movement of the atoms (S412). The processing circuit executes this determination by, for example, determining, from a bonding state change or the like, whether or not a reaction could be appropriately traced. In the case where, for example, the bonding state has stopped in the middle, the processing circuit determines that the movement of the atoms has not been finished.

The processing circuit may determine whether or not an intended chemical reaction could be traced, when, for example, the reaction path search on PES where the artificial force is applied is finished. Several nonlimiting usable examples of a method to determine whether the trace succeeded are as follows.

The processing circuit may determine the presence/absence of the maximum point in the path search with the artificial force being removed and based on the determination result, execute the determination in S412.

The processing circuit may execute the determination by using a typical bond length to compare a symmetric matrix storing bond information before/after the path search. For example, the symmetric matrix may be a matrix that is 1 in the case where the length is less than the typical length, and otherwise, is 0.

The processing circuit may execute the determination in S412 by using RMSD (Root Mean Square Deviation) of the atomic positions in the structures before/after the reaction and comparing it with a reference value.

In the case where the movement of the atoms has not finished (S412: NO), the processing circuit iterates the processes from S404. Here, instead of iterating the processes with the same parameter, the processing circuit may, for example, increase the value of γ to update the parameter to a parameter having a possibility of exceeding the maximum point, and iterate the processes from S404.

The processing circuit selects the coordinates of the atoms where the energy with the artificial force removed becomes maximum (approximate transition state) (S414).

The processing circuit searches for a transition state, starting from the approximate transition state selected in S414 (S416). The processing circuit may use a trained model to execute at least one of the processes involved in the search for the transition state.

The processing circuit obtains structure data that is the result of the search in S416 and optimizes the structure in two directions from the transition state, thereby deciding a reaction path (S418).

As described above, the information processing device according to this embodiment achieves high-speed execution also for the AFIR method. For example, by executing the computations in S404, S408, and so on at a high speed using the trained model, the information processing device achieves greatly higher-speed execution of the AFIR method which requires calculating energy and force for many structures.

Further, for example, by executing the process in S402, it is also possible to easily decide the parameter. Further, for example, by executing the determination in S412, it is possible to surely and accurately search for the reaction path.

In learning for obtaining the trained models used in these embodiments, the three-dimensional structure given as the training data does not necessarily have to include the reaction transition state. For example, by giving, as the training data, a three-dimensional structure deviating from the energy minimum structure, which is obtained in a molecular dynamics calculation, it is possible to obtain a trained model that is capable of inferring energy in the reaction transition state, the first derivative of the energy, the second derivative of the energy, or the anharmonicity of the energy. In this case, in creating the training data, the calculation of the reaction transition state is not required, and accordingly, it is possible to prepare a large amount of training data at a high speed, resulting in an enhanced accuracy of the trained model. It should be noted that the trained models in the previously described embodiments may be, for example, a concept including a model that is trained in the above-described manner and is further distilled by a typical method.

Some or all of each device (the information processing device) in the above embodiment may be configured in hardware, or information processing of software (program) executed by, for example, a CPU (Central Processing Unit), GPU (Graphics Processing Unit). In the case of the information processing of software, software that enables at least some of the functions of each device in the above embodiments may be stored in a non-volatile storage medium (non-volatile computer readable medium) such as CD-ROM (Compact Disc Read Only Memory) or USB (Universal Serial Bus) memory, and the information processing of software may be executed by loading the software into a computer. In addition, the software may also be downloaded through a communication network. Further, entire or a part of the software may be implemented in a circuit such as an ASIC (Application Specific Integrated Circuit) or FPGA (Field Programmable Gate Array), wherein the information processing of the software may be executed by hardware.

A storage medium to store the software may be a removable storage media such as an optical disk, or a fixed type storage medium such as a hard disk, or a memory. The storage medium may be provided inside the computer (a main storage device or an auxiliary storage device) or outside the computer.

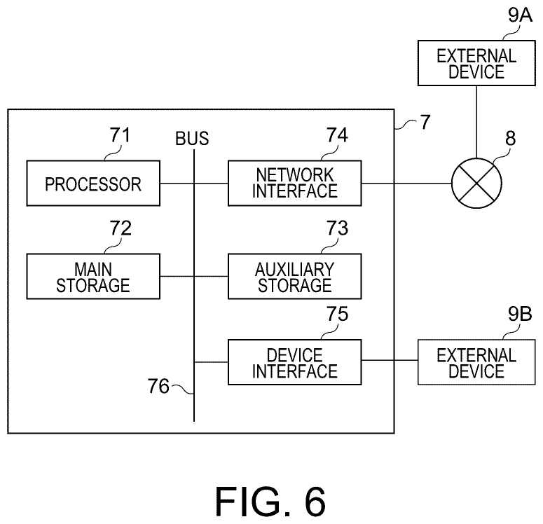

FIG. 6 is a block diagram illustrating an example of a hardware configuration of each device (the information processing device) in the above embodiments. As an example, each device may be implemented as a computer 7 provided with a processor 71, a main storage device 72, an auxiliary storage device 73, a network interface 74, and a device interface 75, which are connected via a bus 76.

The computer 7 of FIG. 6 is provided with each component one by one but may be provided with a plurality of the same components. Although one computer 7 is illustrated in FIG. 6, the software may be installed on a plurality of computers, and each of the plurality of computer may execute the same or a different part of the software processing. In this case, it may be in a form of distributed computing where each of the computers communicates with each of the computers through, for example, the network interface 74 to execute the processing. That is, each device (the information processing device) in the above embodiments may be configured as a system where one or more computers execute the instructions stored in one or more storages to enable functions. Each device may be configured such that the information transmitted from a terminal is processed by one or more computers provided on a cloud and results of the processing are transmitted to the terminal.

Various arithmetic operations of each device (the information processing device) in the above embodiments may be executed in parallel processing using one or more processors or using a plurality of computers over a network. The various arithmetic operations may be allocated to a plurality of arithmetic cores in the processor and executed in parallel processing. Some or all the processes, means, or the like of the present disclosure may be implemented by at least one of the processors or the storage devices provided on a cloud that can communicate with the computer 7 via a network. Thus, each device in the above embodiments may be in a form of parallel computing by one or more computers.

The processor 71 may be an electronic circuit (such as, for example, a processor, processing circuitry, processing circuitry, CPU, GPU, FPGA, or ASIC) that executes at least controlling the computer or arithmetic calculations. The processor 71 may also be, for example, a general-purpose processing circuit, a dedicated processing circuit designed to perform specific operations, or a semiconductor device which includes both the general-purpose processing circuit and the dedicated processing circuit. Further, the processor 71 may also include, for example, an optical circuit or an arithmetic function based on quantum computing.

The processor 71 may execute an arithmetic processing based on data and/or a software input from, for example, each device of the internal configuration of the computer 7, and may output an arithmetic result and a control signal, for example, to each device. The processor 71 may control each component of the computer 7 by executing, for example, an OS (Operating System), or an application of the computer 7.

Each device (the information processing device) in the above embodiments may be enabled by one or more processors 71. The processor 71 may refer to one or more electronic circuits located on one chip, or one or more electronic circuitries arranged on two or more chips or devices. In the case of a plurality of electronic circuitries is used, each electronic circuit may communicate by wired or wireless.

The main storage device 72 may store, for example, instructions to be executed by the processor 71 or various data, and the information stored in the main storage device 72 may be read out by the processor 71. The auxiliary storage device 73 is a storage device other than the main storage device 72. These storage devices shall mean any electronic component capable of storing electronic information and may be a semiconductor memory. The semiconductor memory may be either a volatile or non-volatile memory. The storage device for storing various data or the like in each device (the information processing device) in the above embodiments may be enabled by the main storage device 72 or the auxiliary storage device 73 or may be implemented by a built-in memory built into the processor 71. For example, the storages in the above embodiments may be implemented in the main storage device 72 or the auxiliary storage device 73.

In the case of each device (the information processing device) in the above embodiments is configured by at least one storage device (memory) and at least one processor connected/coupled to/with this at least one storage device, the at least one processor may be connected to a single storage device. Or the at least one storage may be connected to a single processor. Or each device may include a configuration where at least one of the plurality of processors is connected to at least one of the plurality of storage devices. Further, this configuration may be implemented by a storage device and a processor included in a plurality of computers. Moreover, each device may include a configuration where a storage device is integrated with a processor (for example, a cache memory including an L1 cache or an L2 cache).

The network interface 74 is an interface for connecting to a communication network 8 by wireless or wired. The network interface 74 may be an appropriate interface such as an interface compatible with existing communication standards. With the network interface 74, information may be exchanged with an external device 9A connected via the communication network 8. Note that the communication network 8 may be, for example, configured as WAN (Wide Area Network), LAN (Local Area Network), or PAN (Personal Area Network), or a combination of thereof, and may be such that information can be exchanged between the computer 7 and the external device 9A. The internet is an example of WAN, IEEE802.11 or Ethernet (registered trademark) is an example of LAN, and Bluetooth (registered trademark) or NFC (Near Field Communication) is an example of PAN.

The device interface 75 is an interface such as, for example, a USB that directly connects to the external device 9B.

The external device 9A is a device connected to the computer 7 via a network. The external device 9B is a device directly connected to the computer 7.

The external device 9A or the external device 9B may be, as an example, an input device. The input device is, for example, a device such as a camera, a microphone, a motion capture, at least one of various sensors, a keyboard, a mouse, or a touch panel, and gives the acquired information to the computer 7. Further, it may be a device including an input unit such as a personal computer, a tablet terminal, or a smartphone, which may have an input unit, a memory, and a processor.

The external device 9A or the external device 9B may be, as an example, an output device. The output device may be, for example, a display device such as, for example, an LCD (Liquid Crystal Display), or an organic EL (Electro Luminescence) panel, or a speaker which outputs audio. Moreover, it may be a device including an output unit such as, for example, a personal computer, a tablet terminal, or a smartphone, which may have an output unit, a memory, and a processor.

Further, the external device 9A or the external device 9B may be a storage device (memory). The external device 9A may be, for example, a network storage device, and the external device 9B may be, for example, an HDD storage.

Furthermore, the external device 9A or the external device 9B may be a device that has at least one function of the configuration element of each device (the information processing device) in the above embodiments. That is, the computer 7 may transmit a part of or all of processing results to the external device 9A or the external device 9B, or receive a part of or all of processing results from the external device 9A or the external device 9B.

In the present specification (including the claims), the representation (including similar expressions) of “at least one of a, b, and c” or “at least one of a, b, or c” includes any combinations of a, b, c, a-b, a-c, b-c, and a-b-c. It also covers combinations with multiple instances of any element such as, for example, a-a, a-b-b, or a-a-b-b-c-c. It further covers, for example, adding another element d beyond a, b, and/or c, such that a-b-c-d.

In the present specification (including the claims), the expressions such as, for example, “data as input,” “using data,” “based on data,” “according to data,” or “in accordance with data” (including similar expressions) are used, unless otherwise specified, this includes cases where data itself is used, or the cases where data is processed in some ways (for example, noise added data, normalized data, feature quantities extracted from the data, or intermediate representation of the data) are used. When it is stated that some results can be obtained “by inputting data,” “by using data,” “based on data,” “according to data,” “in accordance with data” (including similar expressions), unless otherwise specified, this may include cases where the result is obtained based only on the data, and may also include cases where the result is obtained by being affected factors, conditions, and/or states, or the like by other data than the data. When it is stated that “output/outputting data” (including similar expressions), unless otherwise specified, this also includes cases where the data itself is used as output, or the cases where the data is processed in some ways (for example, the data added noise, the data normalized, feature quantity extracted from the data, or intermediate representation of the data) is used as the output.

In the present specification (including the claims), when the terms such as “connected (connection)” and “coupled (coupling)” are used, they are intended as non-limiting terms that include any of “direct connection/coupling,” “indirect connection/coupling,” “electrical connection/coupling,” “communicative connection/coupling,” “operative connection/coupling,” “physical connection/coupling,” or the like. The terms should be interpreted accordingly, depending on the context in which they are used, but any forms of connection/coupling that are not intentionally or naturally excluded should be construed as included in the terms and interpreted in a non-exclusive manner.

In the present specification (including the claims), when the expression such as “A configured to B,” this may include that a physically structure of A has a configuration that can execute operation B, as well as a permanent or a temporary setting/configuration of element A is configured/set to actually execute operation B. For example, when the element A is a general-purpose processor, the processor may have a hardware configuration capable of executing the operation B and may be configured to actually execute the operation B by setting the permanent or the temporary program (instructions). Moreover, when the element A is a dedicated processor, a dedicated arithmetic circuit, or the like, a circuit structure of the processor or the like may be implemented to actually execute the operation B, irrespective of whether or not control instructions and data are actually attached thereto.

In the present specification (including the claims), when a term referring to inclusion or possession (for example, “comprising/including,” “having,” or the like) is used, it is intended as an open-ended term, including the case of inclusion or possession an object other than the object indicated by the object of the term. If the object of these terms implying inclusion or possession is an expression that does not specify a quantity or suggests a singular number (an expression with a or an article), the expression should be construed as not being limited to a specific number.

In the present specification (including the claims), although when the expression such as “one or more,” “at least one,” or the like is used in some places, and the expression that does not specify a quantity or suggests a singular number (the expression with a or an article) is used elsewhere, it is not intended that this expression means “one.” In general, the expression that does not specify a quantity or suggests a singular number (the expression with a or an as article) should be interpreted as not necessarily limited to a specific number.

In the present specification, when it is stated that a particular configuration of an example results in a particular effect (advantage/result), unless there are some other reasons, it should be understood that the effect is also obtained for one or more other embodiments having the configuration. However, it should be understood that the presence or absence of such an effect generally depends on various factors, conditions, and/or states, etc., and that such an effect is not always achieved by the configuration. The effect is merely achieved by the configuration in the embodiments when various factors, conditions, and/or states, etc., are met, but the effect is not always obtained in the claimed invention that defines the configuration or a similar configuration.

In the present specification (including the claims), when the term such as “maximize/maximization” is used, this includes finding a global maximum value, finding an approximate value of the global maximum value, finding a local maximum value, and finding an approximate value of the local maximum value, should be interpreted as appropriate accordingly depending on the context in which the term is used. It also includes finding on the approximated value of these maximum values probabilistically or heuristically. Similarly, when the term such as “minimize/minimization” is used, this includes finding a global minimum value, finding an approximated value of the global minimum value, finding a local minimum value, and finding an approximated value of the local minimum value, and should be interpreted as appropriate accordingly depending on the context in which the term is used. It also includes finding the approximated value of these minimum values probabilistically or heuristically. Similarly, when the term such as “optimize/optimization” is used, this includes finding a global optimum value, finding an approximated value of the global optimum value, finding a local optimum value, and finding an approximated value of the local optimum value, and should be interpreted as appropriate accordingly depending on the context in which the term is used. It also includes finding the approximated value of these optimal values probabilistically or heuristically.

In the present specification (including claims), when a plurality of hardware performs a predetermined process, the respective hardware may cooperate to perform the predetermined process, or some hardware may perform all the predetermined process. Further, a part of the hardware may perform a part of the predetermined process, and the other hardware may perform the rest of the predetermined process. In the present specification (including claims), when an expression (including similar expressions) such as “one or more hardware perform a first process and the one or more hardware perform a second process,” or the like, is used, the hardware that perform the first process and the hardware that perform the second process may be the same hardware, or may be the different hardware. That is: the hardware that perform the first process and the hardware that perform the second process may be included in the one or more hardware. Note that, the hardware may include an electronic circuit, a device including the electronic circuit, or the like.

In the present specification (including the claims), when a plurality of storage devices (memories) store data, an individual storage device among the plurality of storage devices may store only a part of the data or may store the entire data. Further, some storage devices among the plurality of storage devices may include a configuration for storing data.

While certain embodiments of the present disclosure have been described in detail above, the present disclosure is not limited to the individual embodiments described above. Various additions, changes, substitutions, partial deletions, etc. are possible to the extent that they do not deviate from the conceptual idea and purpose of the present disclosure derived from the contents specified in the claims and their equivalents. For example, when numerical values or mathematical formulas are used in the description in the above-described embodiments, they are shown for illustrative purposes only and do not limit the scope of the present disclosure. Further, the order of each operation shown in the embodiments is also an example, and does not limit the scope of the present disclosure.

1. An information processing device comprising:
one or more memories; and
one or more processors configured to
search for a reaction path by using one or more trained models that, when receiving an input of a three-dimensional arrangement of two or more atoms forming a molecule, output a physical quantity regarding the molecule.

2. The information processing device according to claim 1,
wherein the one or more trained models are models which output at least one of energy, a first derivative of the energy, a second derivative of the energy, a physical quantity from which the second derivative of the energy is calculatable, or anharmonicity.

3. The information processing device according to claim 1,
wherein the trained model is a model trained using training data not containing a reaction transition state.

4. The information processing device according to claim 1,
wherein the one or more processors search for a transition state by using an Anharmonic Downward Distortion Following (ADDF) method.

5. The information processing device according to claim 4,
wherein, in the ADDF method, the one or more processors obtain a Hessian matrix representing a second derivative of energy by using at least one of the one or more trained models, every time molecular deformation is executed.

6. The information processing device according to claim 1,
wherein the one or more processors search for a transition state by using an Artificial Force Induced Reaction (AFIR) method.

7. The information processing device according to claim 6,
wherein the one or more processors decide an artificial force parameter in the AFIR method based on an input parameter.

8. The information processing device according to claim 6,
wherein the one or more processors:
determine whether a search for an intended chemical reaction has been successful, after executing calculation in the AFIR method; and
update, when continuing the search, an artificial force parameter, and execute the path search.

9. The information processing device according to claim 1,
wherein the one or more processors input, into the one or more trained models, the input of the three-dimensional arrangement of two or more atoms forming the molecule, and search for the reaction path based on the physical quantity regarding the molecule output from the one or more trained models.

10. An information processing method, comprising
searching, by one or more processors, for a reaction path by using one or more trained models which, when receiving an input of a three-dimensional arrangement of two or more atoms forming a molecule, output a physical quantity regarding the molecule.

11. A non-transitory computer readable medium storing a program causing one or more processors to execute an information processing method, the information processing method comprising
searching for a reaction path by using one or more trained models which, when receiving an input of a three-dimensional arrangement of two or more atoms forming a molecule, output a physical quantity regarding the molecule.
